(19) United States
(12) Patent Application Publication
(10) Pub. No.: US 2024/0165392 A1
(43) Pub. Date: May 23, 2024

(54) GUIDEWIRE INSERTION AIDES FOR PERCUTANEOUS CIRCULATORY SUPPORT DEVICES

(71) Applicant: Boston Scientific Scimed Inc., Maple Grove, MN (US)

(72) Inventors: Qian Liu, Plymouth, MN (US); Brian R. Reynolds, Ramsey, MN (US); Brice Lee Shireman, Maple Grove, MN (US); Timothy A. Ostroot, Cokato, MN (US)

(21) Appl. No.: 18/385,700

(22) Filed: Oct. 31, 2023

Related U.S. Application Data
(60) Provisional application No. 63/384,333, filed on Nov. 18, 2022.

(51) Int. Cl. A61M 60/13; A61M 60/216; A61M 60/865

(52) U.S. Cl. CPC A61M 60/13; A61M 60/216; A61M 60/865

(57) ABSTRACT

A percutaneous circulatory support system includes a percutaneous circulatory support device. The device includes a housing, an impeller carried in the housing, and a guidewire insertion aide removably carried in the housing. The guidewire insertion aide lacks a through lumen.

### CROSS-REFERENCE TO RELATED PATENT APPLICATIONS

This application claims priority to U.S. Provisional Application No. 63/384,333, filed Nov. 18, 2022, which is herein incorporated by reference in its entirety.

### TECHNICAL FIELD

The present disclosure relates to percutaneous circulatory support devices and associated components. More specifically, the present disclosure relates to guidewire insertion aides for percutaneous circulatory support devices.

### BACKGROUND

Percutaneous circulatory support devices such as blood pumps can provide transient support for up to approximately several weeks in patients with compromised heart function or cardiac output. Such devices are typically delivered to a patient's heart using a guidewire. More specifically, a distal end of the guidewire is inserted into the patient and positioned in the patient's heart while a proximal end of the guidewire remains outside of the patient. The proximal end of the guidewire is then inserted through a percutaneous circulatory support device such that the device is translatable along the guidewire to the patient's heart. However, inserting the guidewire through the percutaneous circulatory support device can damage delicate components of the device.

### SUMMARY

In an Example 1, a percutaneous circulatory support system includes: a percutaneous circulatory support device, including: a housing; an impeller carried in the housing; and a guidewire insertion aide configured to be removably carried in the housing, the guidewire insertion aide lacking a through lumen.

In an Example 2, the percutaneous circulatory support system of Example 1, wherein the guidewire insertion aide is configured to translatably couple to a guidewire.

In an Example 3, the percutaneous circulatory support system of Example 2, wherein the guidewire insertion aide includes a coupler for translatably coupling to the guidewire.

In an Example 4, the percutaneous circulatory support system of Example 2, wherein the percutaneous circulatory support device includes a blood inlet and a blood outlet, and the guidewire insertion aide is configured to be removed from the housing through the blood outlet to pull the guidewire through the blood outlet.

In an Example 5, the percutaneous circulatory support system of Example 4, wherein the guidewire insertion aide includes a proximal end portion and a distal end portion, and the distal end portion is disposed distally relatively to the blood inlet.

In an Example 6, the percutaneous circulatory support system of any of Examples 4-5, wherein the proximal end portion of the guidewire insertion aide is disposed outwardly from the blood outlet.

In an Example 7, a percutaneous circulatory support system includes a percutaneous circulatory support device, including: a housing; an impeller carried in the housing; and a guidewire insertion aide configured to be removably carried in the housing; wherein the guidewire insertion aide is configured to be translatably coupled to a guidewire.

In an Example 8, the percutaneous circulatory support system of Example 7, wherein the guidewire insertion aide includes a coupler for translatably coupling to the guidewire.

In an Example 9, the percutaneous circulatory support system of any of Examples 7-8, wherein the percutaneous circulatory support device includes a blood inlet and a blood outlet, and the guidewire insertion aide is configured to be removed from the housing through the blood outlet to pull the guidewire through the blood outlet.

In an Example 10, the percutaneous circulatory support system of Example 9, wherein the guidewire insertion aide includes a proximal end portion and a distal end portion, and the distal end portion is disposed distally relatively to the blood inlet.

In an Example 11, the percutaneous circulatory support system of any of Examples 9-10, wherein the proximal end portion of the guidewire insertion aide is disposed outwardly from the blood outlet.

In an Example 12, a method of manufacturing a percutaneous circulatory support system includes: providing a percutaneous circulatory support device, the percutaneous circulatory support device including a housing and an impeller carried in the housing; providing a guidewire insertion aide, the guidewire insertion aide lacking a through lumen; and removably positioning the guidewire insertion aide in the housing of the percutaneous circulatory support device.

In an Example 13, the method of Example 12, wherein the guidewire insertion aide includes a coupler for translatably coupling to a guidewire.

In an Example 14, the method of any of Examples 12-13, further including providing the guidewire detached from the guidewire insertion aide.

In an Example 15, the method of any of Examples 12-14, wherein the percutaneous circulatory support device further includes a cannula coupled to the housing, and further including removably positioning the guidewire insertion aide in the cannula.

In an Example 16, a percutaneous circulatory support system includes: a percutaneous circulatory support device, including: a housing; an impeller carried in the housing; and a guidewire insertion aide configured to be removably carried in the housing; wherein the guidewire insertion aide is configured to be translatably coupled to a guidewire.

In an Example 17, the percutaneous circulatory support system of Example 16, wherein the guidewire insertion aide includes a coupler for translatably coupling to the guidewire.

In an Example 18, the percutaneous circulatory support system of Example 16, wherein the percutaneous circulatory support device includes a blood inlet and a blood outlet, and the guidewire insertion aide is configured to be removed from the housing through the blood outlet to pull the guidewire through the blood outlet.

In an Example 19, the percutaneous circulatory support system of Example 18, wherein the guidewire insertion aide includes a proximal end portion and a distal end portion, and the distal end portion is disposed distally relatively to the blood inlet.

In an Example 20, the percutaneous circulatory support system of Example 4, wherein the proximal end portion of the guidewire insertion aide is disposed outwardly from the blood outlet.

In an Example 21, the percutaneous circulatory support system of Example 16, wherein the guidewire insertion aide lacks a through lumen.

In an Example 22, a percutaneous circulatory support system includes: a percutaneous circulatory support device, including: a housing; an impeller carried in the housing; a guidewire insertion aide removably carried in the housing; and a guidewire; wherein the guidewire insertion aide and the guidewire are configured to be translatably coupled such that subsequent removal of the guidewire insertion aide from the housing causes the guidewire to be pulled through the housing.

In an Example 23, the percutaneous circulatory support system of Example 22, wherein the percutaneous circulatory support device includes a blood inlet and a blood outlet, and the guidewire insertion aide is configured to be removed from the housing through the blood outlet to pull the guidewire through the blood outlet.

In an Example 24, the percutaneous circulatory support system of Example 23, wherein the guidewire insertion aide includes a proximal end portion and a distal end portion, and the distal end portion is disposed distally relatively to the blood inlet.

In an Example 25, the percutaneous circulatory support system of Example 23, wherein the proximal end portion of the guidewire insertion aide is disposed outwardly from the blood outlet.

In an Example 26, the percutaneous circulatory support system of Example 22, wherein the guidewire insertion aide includes a coupler for translatably coupling to the guidewire.

In an Example 27, the percutaneous circulatory support system of Example 7, wherein the guidewire includes a coupler for translatably coupling to the guidewire insertion aide.

In an Example 28, the percutaneous circulatory support system of Example 22, wherein the guidewire insertion aide lacks a through lumen.

In an Example 29, a method of manufacturing a percutaneous circulatory support system includes: providing a percutaneous circulatory support device, the percutaneous circulatory support device including a housing and an impeller carried in the housing; providing a guidewire insertion aide, the guidewire insertion aide being configured to be translatably secured to a guidewire; and removably positioning the guidewire insertion aide in the housing of the percutaneous circulatory support device.

In an Example 30, the method of Example 29, wherein the guidewire insertion aide includes a coupler for translatably coupling to a guidewire.

In an Example 31, the method of Example 29, wherein the guidewire insertion aide lacks a through lumen.

In an Example 32, the method of Example 29, further including providing the guidewire detached from the guidewire insertion aide.

In an Example 33, the method of Example 32, wherein the guidewire includes a coupler for translatably coupling to the guidewire insertion aide.

In an Example 34, the method of Example 29, wherein the percutaneous circulatory support device further includes a cannula coupled to the housing, and further including removably positioning the guidewire insertion aide in the cannula.

In an Example 35, the method of Example 34, wherein the percutaneous circulatory support device further includes a flexible distal tip portion coupled to the cannula opposite the housing, and further including removably positioning the guidewire insertion aide in the flexible distal tip portion.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a side sectional view of an illustrative percutaneous circulatory support system including a percutaneous circulatory support device (also referred to herein, interchangeably, as a “blood pump”) and a guidewire insertion aide, in accordance with embodiments of the subject matter disclosed herein.

FIG. 2 is a side view of the guidewire insertion aide of FIG. 1 and an illustrative guidewire, in accordance with embodiments of the subject matter disclosed herein.

FIG. 3 is a side view of another guidewire insertion aide, in accordance with embodiments of the subject matter disclosed herein.

FIG. 4 is a side view of another guidewire insertion aide, in accordance with embodiments of the subject matter disclosed herein.

FIG. 5 is a side view of another guidewire insertion aide, in accordance with embodiments of the subject matter disclosed herein.

FIG. 6 is a side view of another guidewire insertion aide, in accordance with embodiments of the subject matter disclosed herein.

FIG. 7 is a side view of another guidewire insertion aide, in accordance with embodiments of the subject matter disclosed herein.

FIG. 8 is a side view of another guidewire insertion aide in an initial configuration, in accordance with embodiments of the subject matter disclosed herein.

FIG. 9 is another side view of the guidewire insertion aide of FIG. 8 in a coupling configuration, being translatably coupled to a guidewire.

FIG. 10 is a side view of another guidewire insertion aide, in accordance with embodiments of the subject matter disclosed herein.

FIG. 11 is a side view of another guidewire insertion aide, in accordance with embodiments of the subject matter disclosed herein.

FIG. 12 is a side sectional view of yet another guidewire insertion aide, in accordance with embodiments of the subject matter disclosed herein.

FIG. 13 is a side view of the guidewire insertion aide of FIG. 12.

FIG. 14 is a diagram of a method of manufacturing a percutaneous circulatory support system, in accordance with embodiments of the subject matter disclosed herein.

FIG. 15 is a diagram of a method of using a percutaneous circulatory support system, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

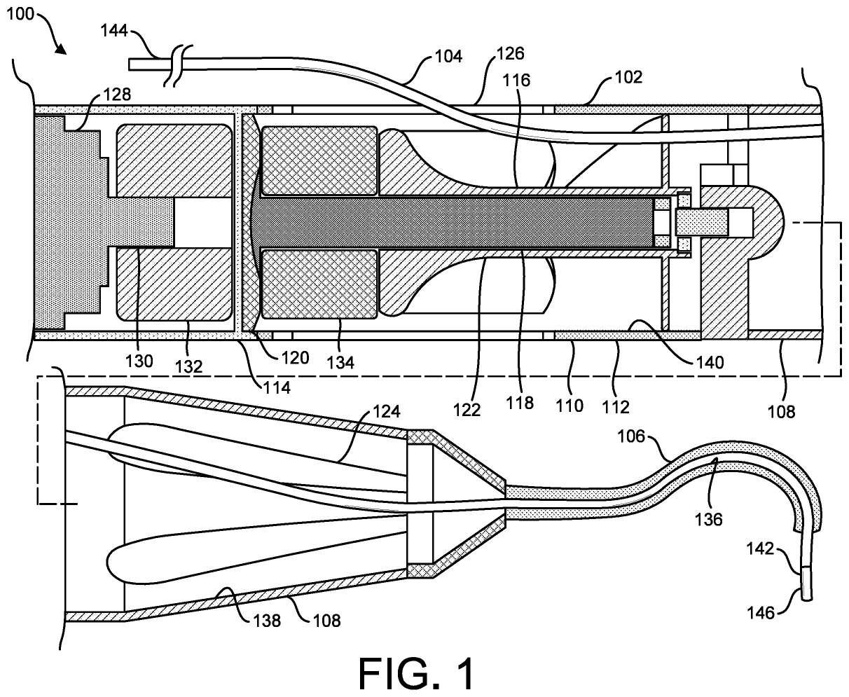

FIG. 1 depicts a partial side sectional view of an illustrative percutaneous circulatory support system 100 in accordance with embodiments of the subject matter disclosed herein. The system 100 includes a percutaneous circulatory support device 102 (also referred to herein, interchangeably, as a “blood pump”), a guidewire insertion aide 104, a guidewire (shown elsewhere), and an introducer sheath (not shown). The guidewire insertion aide 104 initially extends through the device 102 (that is, the guidewire insertion aide 104 extends through the device 102 before the system 100 is provided to a medical practitioner) and, as described in further detail below, facilitates inserting the guidewire into the device 102 without potentially damaging delicate components of the device 102. After inserting the guidewire into the device 102, the guidewire and the introducer sheath may be used to percutaneously deliver the device 102 to a target location within a patient. More specifically, the device 102 may be translated along the guidewire to deliver the device 102 to the target location. The target location may be within the patient's heart. Alternatively, the device 102 may be delivered to a different target location within a patient. The introducer sheath and/or other devices may be used to facilitate delivery of device 102.

With continued reference to FIG. 1, the device 102 generally includes a flexible distal tip portion 106, a cannula 108, an impeller portion 110, and a proximal catheter (not shown). During operation, the device 102 may be positioned such that the distal tip portion 106 is located in close proximity of, or in contact with, the wall of the left ventricle of the patient, for example, in the location of the apex of the left ventricle. The cannula 108 may be partially positioned in the left ventricle and extend through the aortic valve of the patient. The impeller portion 110 and the catheter may be positioned in the aorta of the patient.

In some embodiments, the impeller portion 110 includes an impeller housing 112 and a motor housing 114. The impeller housing 112 and the motor housing 114 may be integrally or monolithically constructed. In other embodiments, the impeller housing 112 and the motor housing 114 may be separate components configured to be removably or permanently coupled. In other embodiments, the device 102 may lack the motor housing 114.

The impeller housing 112 carries an impeller assembly 116 therein. The impeller assembly 116 includes an impeller shaft 118 that is rotatably supported by at least one bearing, such as a bearing 120. The impeller assembly 116 also includes an impeller 122 that rotates relative to the impeller housing 112 to drive blood through the device 102. More specifically, the impeller 122 causes blood to flow from a blood inlet 124 of the cannula 108, through the cannula 108 and the impeller housing 112, and out of a blood outlet 126 formed on the impeller housing 112. In some embodiments and as illustrated, the impeller shaft 118 and the impeller 122 may be separate components, and in other embodiments the impeller shaft 118 and the impeller 122 may be integrated. In some embodiment and as illustrated, the inlet 124 and/or the outlet 126 may each include multiple apertures. In other embodiments, the inlet 124 and/or the outlet 126 may each include a single aperture. In some embodiments and as illustrated, the inlet 124 may be formed on a side portion of the cannula 108 and the outlet 126 may be formed on a side portion of the impeller housing 112.

With continued reference to FIG. 1, the motor housing 114 carries a motor 128, and the motor 128 is configured to rotatably drive the impeller 122 relative to the impeller housing 112. In the illustrated embodiment, the motor 128 rotates a drive shaft 130, which is coupled to a driving magnet 132. Rotation of the driving magnet 132 causes rotation of a driven magnet 134, which is connected to and rotates together with the impeller assembly 116. More specifically, in embodiments incorporating the impeller shaft 118, the impeller shaft 118 and the impeller 122 are configured to rotate with the driven magnet 134. In other embodiments, the motor 128 may couple to the impeller assembly 116 via other components. As noted above, rotation of the impeller 122 causes blood to flow through the device 102.

In some embodiments, a controller (not shown) may be operably coupled to the motor 128 and configured to control the motor 128. In some embodiments, the controller may be disposed within the motor housing 114. In other embodiments, the controller may be disposed outside of the motor housing 114 (for example, in a catheter handle, an independent housing, etc.). In some embodiments, the controller may include multiple components, one or more of which may be disposed within the motor housing 114. According to embodiments, the controller may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller is referred to herein in the singular, the controller may be implemented in multiple instances, distributed across multiple computing devices, instantiated within multiple virtual machines, and/or the like. In other embodiments, the motor 128 may be controlled in other manners.

With further reference to FIG. 1 and as described briefly above, the guidewire insertion aide 104 initially extends through the device 102. More specifically, the guidewire insertion aide 104 extends through a lumen 136 of the distal tip portion 106, through a lumen 138 of the cannula 108, into an interior 140 of the impeller housing 112, and out of the blood outlet 126 of the impeller housing 112. Accordingly, a distal end portion 142 of the guidewire insertion aide 104 may be initially disposed distally relative to the distal tip portion 106, and an opposite proximal end portion 144 of the guidewire insertion aide 104 may be initially disposed proximally relative to the blood outlet 126. In some embodiments, such as embodiments in which the device 102 lacks the distal tip portion 106, the guidewire insertion aide 104 may extend into the blood inlet 124 of the cannula 108, through the cannula 108, into the interior 140 of the impeller housing 112, and out of the blood outlet 126 of the impeller housing 112. In such embodiments, the distal end portion 142 of the guidewire insertion aide 104 maybe initially disposed distally relative to the blood inlet 124 of the cannula 108.

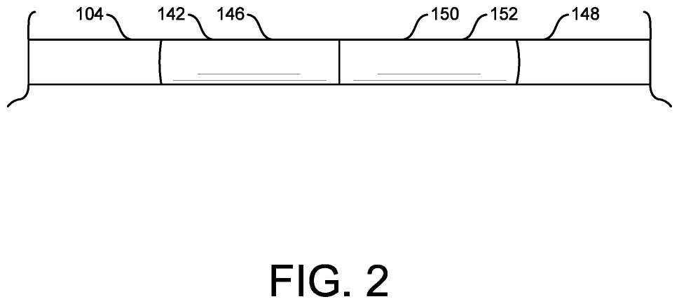

With reference now to FIGS. 1 and 2, the guidewire insertion aide 104 may be, generally, an elongated, flexible wire or cable that is suitable for pulling a guidewire into the device 102. The guidewire insertion aide 104 may be constructed of various appropriate materials, such as relatively soft biocompatible metals, polymers, composites, or the like. In some embodiments, the guidewire insertion aide 104 or a portion thereof is sufficiently flexible such that the guidewire insertion aide 104 does not initially deform the distal tip portion 106 of the device 102. For example, in some embodiments the guidewire insertion aide 104 may be constructed such that it can be heat-set or shaped to maintain a shape of the distal tip portion 106. The desired flexibility may be achieved by varying the shape along the guidewire insertion aide 104, constructing the guidewire insertion 104 of various materials, or by other known means. In some embodiments, a distal portion of the guidewire insertion aide 104 may constructed differently than a proximal portion of the guidewire insertion aide 104 to achieve the desired flexibility in the distal portion.

With continued reference to FIG. 2, the guidewire insertion aide 104 may include a coupler 146 for translatably coupling to the guidewire 148 (that is, coupling such that the guidewire insertion aide 104 and the guidewire 148 are translatable together). As illustrated, the coupler 146 may be disposed at the distal end portion 142 of the guidewire insertion aide 104. The coupler 146 may take various forms. For example, the coupler 146 may be or include one or more temporary fasteners, such as a threaded surface, a blind hole (that is, a hole including a single opening), a snap connector, a press-fit connector, or a magnetic connector, one or more permanent fasteners, such a crimp connector, or any of the other structures contemplated herein. Several of these examples, and other examples, are described in further detail below. The guidewire 148 may also include a coupler 150 for translatably coupling to the guidewire insertion aide 104, more specifically coupling to the coupler 146 of the guidewire insertion aide 104. As illustrated, the coupler 150 may be disposed at the proximal end portion 152 of the guidewire 148. The coupler 150 may take various forms. For example, the coupler 150 may be or include one or more temporary fasteners, such as a threaded surface, a blind hole, a snap connector, a press-fit connector, or a magnetic connector, one or more permanent fasteners, such a crimp connector, or any of the other structures contemplated herein. In some embodiments, the guidewire 148 may be a conventional guidewire (that is, the guidewire 148 may lack the coupler 150). In some embodiments, the guidewire 148 may include the coupler 150 and the guidewire insertion aide 104 may lack the coupler 146. It is envisioned that various types of guidewires may be used in the conjunction with the guidewire insertion aide 104. For example, different sizes of guidewires, including guidewires have diameters of 0.014 inches, 0.018 inches, or other commonly sized guidewires may be used with guidewire insertion aide 104. It is also envisioned that different sized guidewire insertion aides may be used to correspond to different sized guidewires. Furthermore, it is envisioned that the coupler 146 may be constructed such that guidewire insertion aide 104 may be coupled to a variety of differently sized guidewires.

In some embodiments and as illustrated, the guidewire insertion aide 104 lacks a through lumen (that is, a lumen including two openings) through which a guidewire may extend. For example, the guidewire insertion aide 104 may include a short lumen that does not extend the entire length of the aide 104 such that the majority of the length of the aide 104 is without a lumen. One example of such an embodiment includes a guidewire insertion aide 104 comprising a loading aide connector tube coupled to a loading aide wire. In such an embodiment, the loading aide connector tube includes a lumen and the loading aide wire does not include a lumen, and the loading aid connector tube is used to couple the loading aid wire to a guidewire.

As illustrated, the guidewire insertion aide 104 may have the same general size (more specifically, a diameter) as the guidewire 148. In other embodiments, the guidewire insertion aide 104 may have a different general size (more specifically, a diameter) than the guidewire 148.

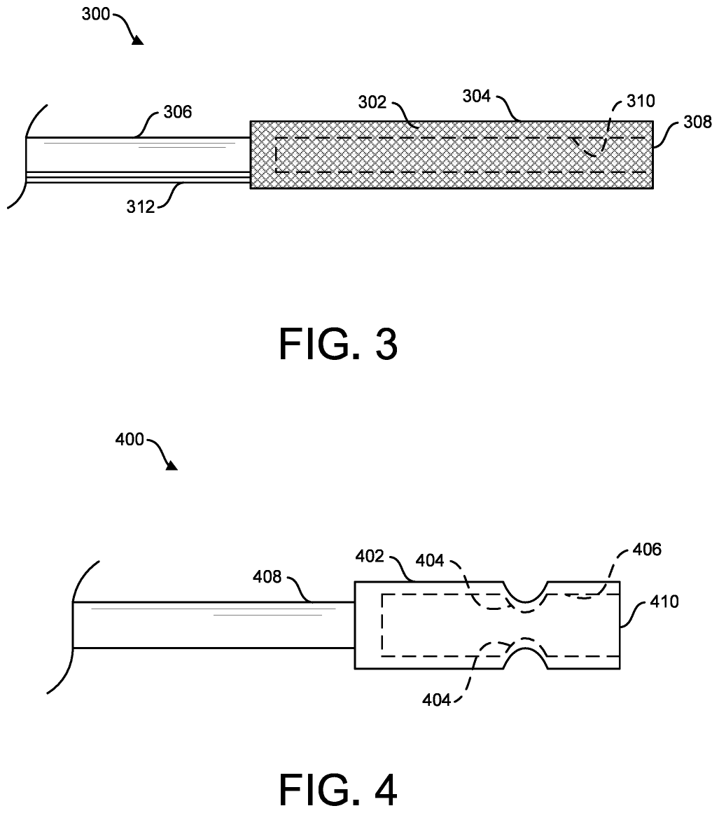

Referring to FIG. 3, a guidewire insertion aide 300 in accordance with embodiments of the subject matter disclosed herein is illustrated. The guidewire insertion aide 300 is configured to couple to a guidewire, such as the guidewire 148 described herein. The guidewire insertion aide 300 includes similar features and is used in a manner similar to the guidewire insertion aide 104 described herein. As a coupler for coupling to a guidewire, the guidewire insertion aide 300 specifically includes a collapsible structure 302, such as a collapsible braided structure. The collapsible structure 302 is encapsulated in an undersized outer sleeve 304, such as a polymer sleeve, and coupled to a main wire 306. By inserting the proximal end of the guidewire into a distal opening 308 and a lumen 310 of the collapsible structure 302, the collapsible structure 302 expands. However, the undersized outer sleeve 304 compresses the collapsible structure 302 onto the guidewire and thereby provides frictional engagement between the collapsible structure 302 and the guidewire. The guidewire is thereby translatably coupled to the guidewire insertion aide 300, and the guidewire may be pulled through a blood pump by pulling on the main wire 306 of the guidewire insertion aide 300. In some embodiments, a coupler of a guidewire may include the collapsible structure 302.

Referring to FIG. 4, a guidewire insertion aide 400 in accordance with embodiments of the subject matter disclosed herein is illustrated. The guidewire insertion aide 400 is configured to couple to a guidewire, such as the guidewire 148 described herein. The guidewire insertion aide 400 includes similar features and is used in a manner similar to the guidewire insertion aide 104 described herein. As a coupler for coupling to a guidewire, the guidewire insertion aide 400 specifically includes a tube 402 having one or more relatively narrow portions 404 (illustratively, two narrow portions 404), or indentations or “pinch points”, within a lumen 406. The tube 402 is coupled to a main wire 408. Upon insertion of the proximal end of the guidewire into a distal opening 410 and the lumen 406 of the tube 402, the narrow portions 404 frictionally engage the guidewire. The guidewire is thereby translatably coupled to the guidewire insertion aide 400, and the guidewire may be pulled through a blood pump by pulling on the main wire 408 of the guidewire insertion aide 400. In some embodiments, a coupler of a guidewire may include the tube 402.

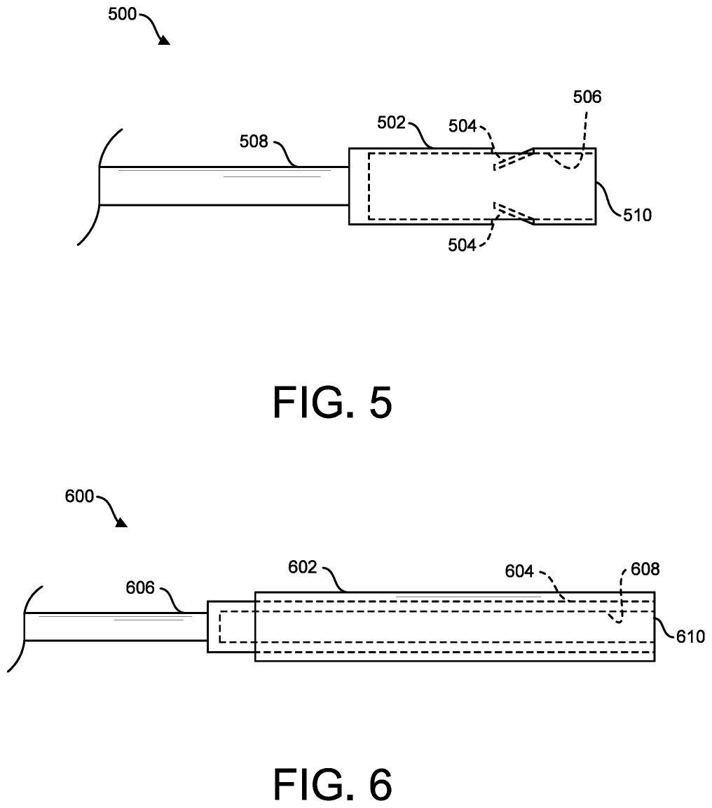

Referring to FIG. 5, a guidewire insertion aide 500 in accordance with embodiments of the subject matter disclosed herein is illustrated. The guidewire insertion aide 500 is configured to couple to a guidewire, such as the guidewire 148 described herein. The guidewire insertion aide 500 includes similar features and is used in a manner similar to the guidewire insertion aide 104 described herein. As a coupler for coupling to a guidewire, the guidewire insertion aide 500 specifically includes a tube 502 having relatively one or more relatively narrow portions 504 (illustratively, two narrow portions 504), or indentations or inwardly bent “fingers”, within a lumen 506. The tube 502 is coupled to a main wire 508. Upon insertion of the proximal end of the guidewire into a distal opening 510 and the lumen 506 of the tube 502, the narrow portions 504 frictionally engage the guidewire. The guidewire is thereby translatably coupled to the guidewire insertion aide 500, and the guidewire may be pulled through a blood pump by pulling on the main wire 508 of the guidewire insertion aide 500. In some embodiments, a coupler of a guidewire may include the tube 502.

Referring to FIG. 6, a guidewire insertion aide 600 in accordance with embodiments of the subject matter disclosed herein is illustrated. The guidewire insertion aide 600 is configured to couple to a guidewire, such as the guidewire 148 described herein. The guidewire insertion aide 600 includes similar features and is used in a manner similar to the guidewire insertion aide 104 described herein. As a coupler for coupling to a guidewire, the guidewire insertion aide 600 specifically includes an outer tube 602 that carries an internal flexible sleeve 604, such as an elastic polymer sleeve. The outer tube 602 and the internal sleeve 604 are coupled to a main wire 606. The diameter of a lumen 608 of the internal sleeve 604 is slightly smaller than the diameter of the guidewire. Upon insertion of the proximal end of the guidewire into a distal opening 610 and the lumen 608 of the internal sleeve 604, the internal sleeve 604 is compressed against the guidewire and frictionally engages the guidewire. The guidewire is thereby translatably coupled to the guidewire insertion aide 600, and the guidewire may be pulled through a blood pump by pulling on the main wire 606 of the guidewire insertion aide 600. In some embodiments, a coupler of a guidewire may include the outer tube 602 and the internal sleeve 604.

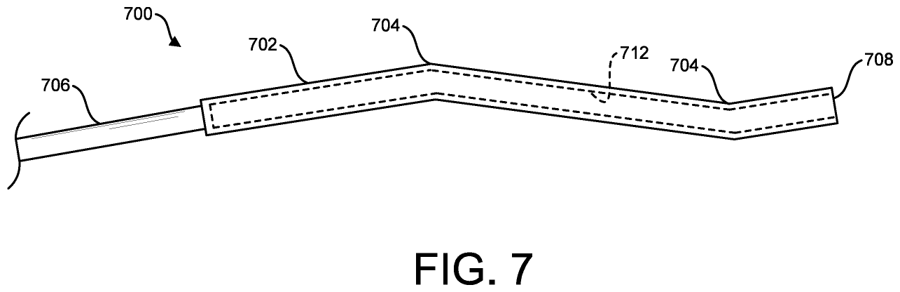

Referring to FIG. 7, a guidewire insertion aide 700 in accordance with embodiments of the subject matter disclosed herein is illustrated. The guidewire insertion aide 700 is configured to couple to a guidewire, such as the guidewire 148 described herein. The guidewire insertion aide 700 includes similar features and is used in a manner similar to the guidewire insertion aide 104 described herein. As a coupler for coupling to a guidewire, the guidewire insertion aide 700 specifically includes a tube 702 having one or more bends 704. Illustratively, the tube 702 includes two bends 704 in a common plane. In other embodiments, the tube 702 may include bends 704 in various planes (that is, the tube 702 may be bent in a three-dimensional manner). In any case, the tube 702 is coupled to a main wire 706. After insertion of the proximal end of the guidewire into a distal opening 708 and a lumen 712 of the tube 702, the main wire 706 is pulled by practitioner and the bends 704 frictionally engage the guidewire. The guidewire is thereby translatably coupled to the guidewire insertion aide 700, and the guidewire may be pulled through a blood pump by pulling on the main wire 706 of the guidewire insertion aide 700. In other embodiments, the tube 702 may initially be straight and, after insertion of the guidewire into the tube 702, the tube 702 may be bent by a practitioner to translatably couple the guidewire to the guidewire insertion aide 700. In some embodiments, a coupler of a guidewire may include the tube 702.

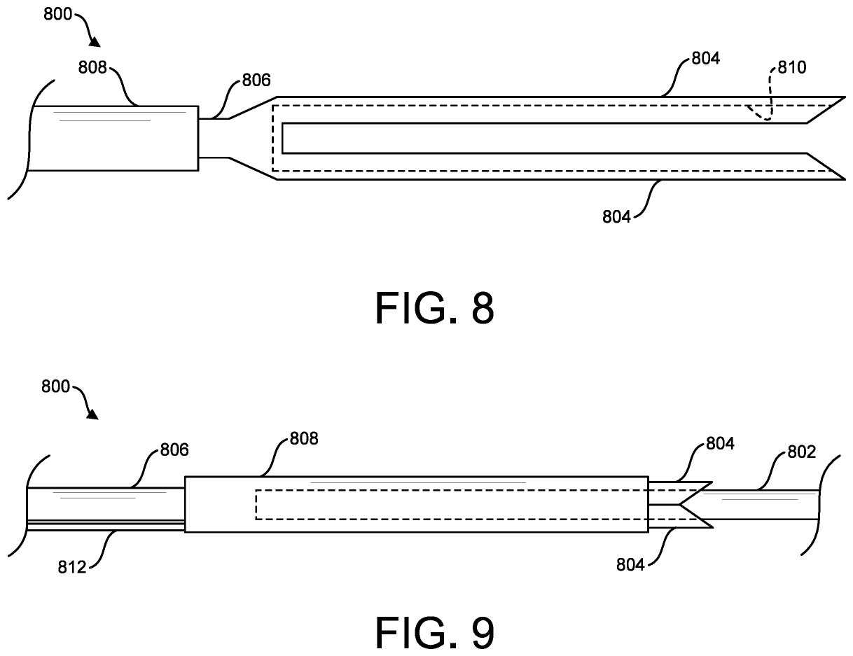

Referring to FIGS. 8 and 9, a guidewire insertion aide 800 in accordance with embodiments of the subject matter disclosed herein is illustrated. FIG. 8 illustrates the guidewire insertion aide 800 in an initial configuration, and FIG. 9 illustrates the guidewire insertion aide 800 in a coupling configuration, in which the aide 800 is coupled to a guidewire 802. The guidewire 802 may be the same or similar to the guidewire 148 described herein. The guidewire insertion aide 800 includes similar features and is used in a manner similar to the guidewire insertion aide 104 described herein. As a coupler for coupling to a guidewire, the guidewire insertion aide 800 specifically includes a plurality of legs 804 (illustratively, two legs 804), a main wire 806, and a slidable collet 808. The legs 804 define a lumen 810 (FIG. 8) therebetween for receiving the guidewire 802. After receiving the guidewire 802 in the lumen 810, an actuator, such as a push wire 812, is actuated to slide the collet 808 over the legs 804 to compress the legs 804 and thereby pinch the guidewire 802. The guidewire 802 is thereby translatably coupled to the guidewire insertion aide 800, and the guidewire 802 may be pulled through a blood pump by pulling on the main wire 806 of the guidewire insertion aide 800. In some embodiments, the legs 804 may include one or more stops (not shown) to inhibit the collet 808 from detaching from the legs 804. In some embodiments, the collet 808 may include textured surfaces and/or larger profiles to facilitate gripping the guidewire 802. In some embodiments, the legs 804 may include textured surfaces to facilitate gripping the guidewire 802. In some embodiments, the collet 808 may be used with a high-force opening/closing tool to facilitate gripping the guidewire 802 and permit ease of re-opening the aide 800. In some embodiments, the aide 800 may include a stop feature (not shown—for example, a wire or mandrel disposed between the legs 804) to limit the coupling interface length with the guidewire 802. In some embodiments, the aide 800 may include a high-contrast differential color scheme to facilitate ease of identification and use of the components of the aide 800. In some embodiments, various edges of the aide 800 may be chamfered or rounded to reduce or eliminate catch points. In some embodiments, a coupler of a guidewire may include the legs 804 and the slidable collet 808.

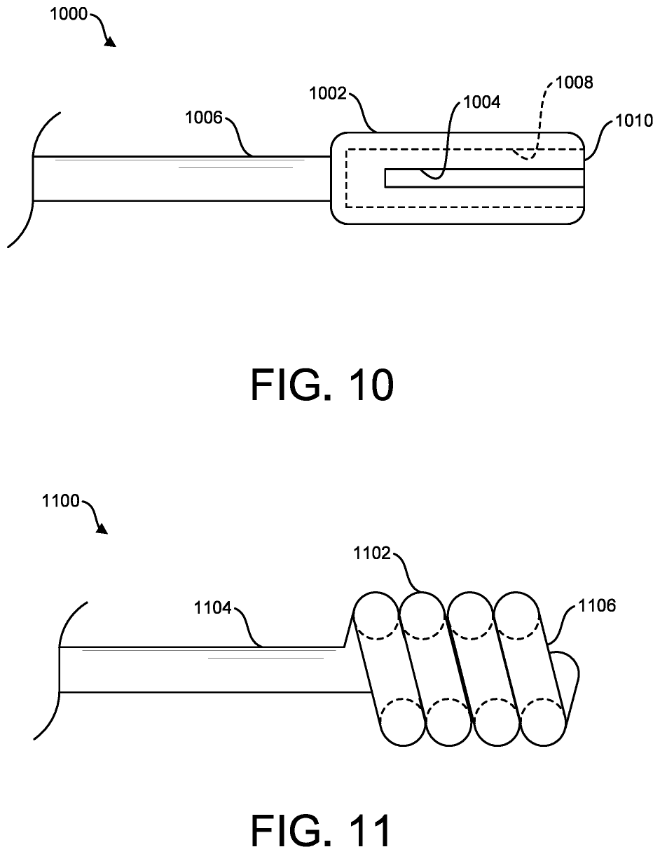

Referring to FIG. 10, a guidewire insertion aide 1000 in accordance with embodiments of the subject matter disclosed herein is illustrated. The guidewire insertion aide 1000 is configured to couple to a guidewire, such as the guidewire 148 described herein. The guidewire insertion aide 1000 includes similar features and is used in a manner similar to the guidewire insertion aide 104 described herein. As a coupler for coupling to a guidewire, the guidewire insertion aide 1000 specifically includes a tube 1002 having one or more longitudinally extending slots 1004 (illustratively, one slot 1004). The tube 1002 is coupled to a main wire 1006. The diameter of a lumen 1008 of the tube 1002 is slightly smaller than the diameter of the guidewire. Upon insertion of the proximal end of the guidewire into a distal opening 1010 and the lumen 1008 of the tube 1002, the tube 1002 spreads apart at the slot 1004 and pinches the guidewire. The guidewire is thereby translatably coupled to the guidewire insertion aide 1000, and the guidewire may be pulled through a blood pump by pulling on the main wire 1006 of the guidewire insertion aide 1000. In some embodiments, a coupler of a guidewire may include the tube 1002.

Referring to FIG. 11, a guidewire insertion aide 1100 in accordance with embodiments of the subject matter disclosed herein is illustrated. The guidewire insertion aide 1100 is configured to couple to a guidewire, such as the guidewire 148 described herein. The guidewire insertion aide 1100 includes similar features and is used in a manner similar to the guidewire insertion aide 104 described herein. As a coupler for coupling to a guidewire, the guidewire insertion aide 1100 specifically includes a coil 1102 coupled to a main wire 1104. The inner diameter of the coil 1102 is slightly smaller than the diameter of the guidewire. Upon insertion of the proximal end of the guidewire into a distal opening 1106 of the coil 1102, the coil 1102 expands outwardly and pinches the guidewire. The guidewire is thereby translatably coupled to the guidewire insertion aide 1100, and the guidewire may be pulled through a blood pump by pulling on the main wire 1104 of the guidewire insertion aide 1100. In some embodiments, a coupler of a guidewire may include the coil 1102.

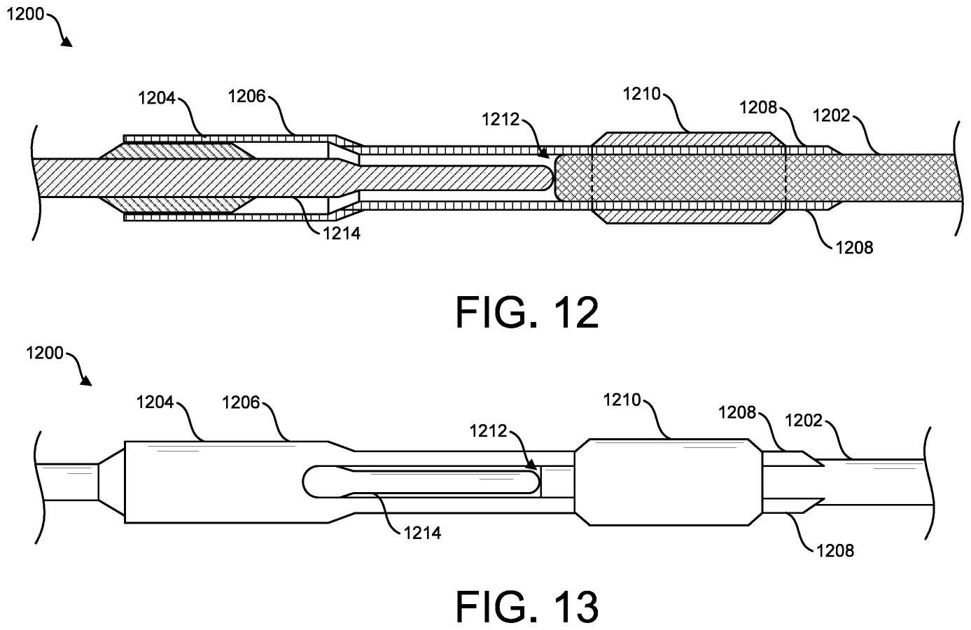

Referring to FIGS. 12 and 13, a low-profile guidewire insertion aide 1200 in accordance with embodiments of the subject matter disclosed herein is illustrated. The guidewire insertion aide 1200 is illustrated in a coupling configuration, in which the aide 1200 is coupled to a guidewire 1202. The guidewire 1202 may be the same or similar to the guidewire 148 described herein. The guidewire insertion aide 1200 includes similar features and is used in a manner similar to the guidewire insertion aide 104 described herein. As a coupler for coupling to a guidewire, the guidewire insertion aide 1200 specifically includes a body 1204 having a base portion 1206 and a plurality of legs 1208 (illustratively, two legs 1208). The coupler also includes a slidable collet 1210. The legs 1208 define a lumen 1212 therebetween for receiving the guidewire 1202. After receiving the guidewire 1202 in the lumen 1212 and preferably contacting the guidewire 1202 against a main wire 1214 of the loading aide 1200, an actuator, such as a push wire (not shown), is actuated to slide the collet 1210 over the legs 1208 to compress the legs 1208 and thereby pinch the guidewire 1202. The guidewire 1202 is thereby translatably coupled to the guidewire insertion aide 1200, and the guidewire 1202 may be pulled through a blood pump by pulling on the main wire 1214 of the guidewire insertion aide 1200. In some embodiments, the base portion 1206 may have an outer diameter greater than the inner diameter of the collet 1210 to inhibit the collet 1210 from translating proximally along the main wire 1214. In some embodiments, the base portion 1206 may have an outer diameter of about 0.021 inches to 0.031 inches, more specifically about 0.026 inches, and the collet 1210 may have an inner diameter of about 0.019 inches to 0.029 inches, more specifically about 0.024 inches. In some embodiments, a coupler of a guidewire may include the body 1204 and the slidable collet 1210.

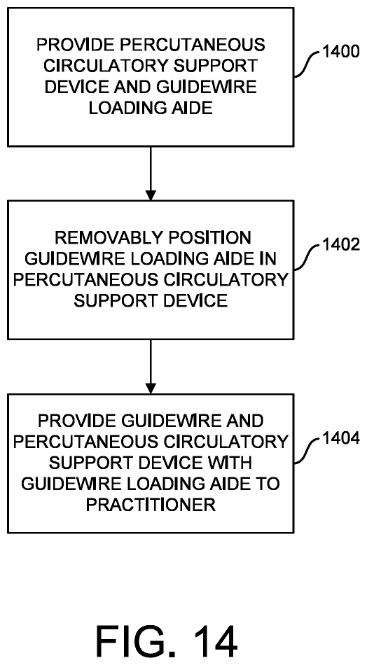

Referring to FIG. 14, a method of assembling a percutaneous circulatory support system in accordance with embodiments of the subject matter disclosed herein may be generally as follows. The method describes features of the system 100, although it is understood that any system contemplated herein could be used in a similar manner. At block 1400, the device 102 and the guidewire insertion aide 104 are provided. At block 1402, the guidewire insertion aide 104 is removably positioned in the impeller housing 112, the cannula 108, and the distal tip portion 106 of the device 102 and disposed, for example, as shown in FIG. 1. The above actions may be conducted in manufacturing environment (that is, not a medical environment). At block 1404, the device 102 with the guidewire insertion aide 104 positioned in the device 102 is packaged for use by a medical practitioner (for example, a surgeon, a nurse, a medical environment administrator, or the like). The guidewire 148, detached from the guidewire insertion aide 104, may also be packaged for use by a medical practitioner.

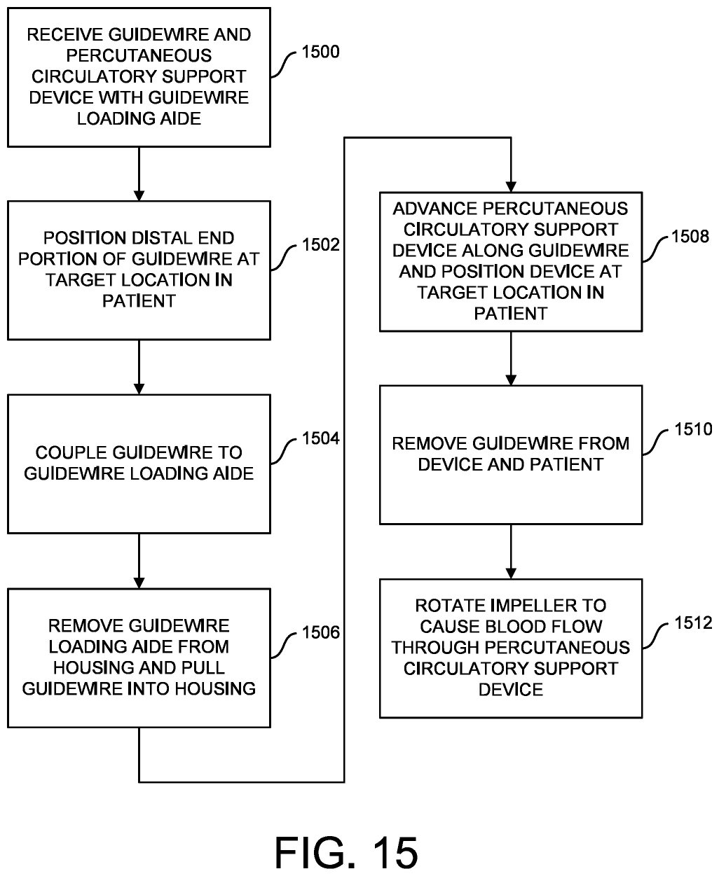

Referring to FIG. 15, a method of using a percutaneous circulatory support system in accordance with embodiments of the subject matter disclosed herein may be generally as follows. The method describes features of the system 100, although it is understood that any system contemplated herein could be used in a similar manner. At block 1500, the device 102 and the guidewire 148 are received by a medical practitioner. Upon receipt of the device 102, the guidewire insertion aide 104 is removably carried by the impeller housing 112 of the device 102 and disposed, for example, as shown in FIG. 1. At block 1502, the distal end portion of the guidewire 148 is inserted in the patient and advanced to the target location in the patient. At block 1504, the guidewire 148 is coupled to the guidewire insertion aide 104, for example, via the coupler 146 at the distal end portion 142 of the guidewire insertion aide 104 and/or the coupler 150 at the proximal end portion 152 of the guidewire 148. At block 1506, the guidewire insertion aide 104 is removed from the impeller housing 112 of the device 102 to pull the guidewire 148 into the impeller housing 112. More specifically, the guidewire insertion aide 104 is pulled out of the blood outlet 126 to pull the proximal end portion 152 of the guidewire 148 through the distal tip portion 106, the cannula 108, the impeller housing 112, and the blood outlet 126. The guidewire insertion aide 104 may then be detached from the guidewire 148, or the guidewire insertion aide 104 may remain coupled to the guidewire 148. At block 1508, the device 102 is advanced along the guidewire 148 and positioned at the target location in the patient. At block 1510, the guidewire 148 is pulled proximally and thereby removed from the device 102 and the patient. At block 1512, the device is energized to rotate the impeller 122 relative to the impeller housing 112 and cause blood to flow through the device 102.

As described above, guidewire insertion aides according to the present disclosure initially extends through percutaneous circulatory support devices and facilitates inserting the guidewire into the devices. Because the guidewire insertion aides are positioned in the devices in a manufacturing environment (that is, not by a medical practitioner) and/or due to the materials of the guidewire insertion aides, embodiments according to the present disclosure avoid damage to delicate components of percutaneous circulatory support devices that may otherwise be caused by guidewire insertion.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A percutaneous circulatory support system, comprising:
a percutaneous circulatory support device, comprising:
a housing;
an impeller carried in the housing; and
a guidewire insertion aide configured to be removably carried in the housing;
wherein the guidewire insertion aide is configured to be translatably coupled to a guidewire.

2. The percutaneous circulatory support system of claim 1, wherein the guidewire insertion aide comprises a coupler for translatably coupling to the guidewire.

3. The percutaneous circulatory support system of claim 1, wherein the percutaneous circulatory support device comprises a blood inlet and a blood outlet, and the guidewire insertion aide is configured to be removed from the housing through the blood outlet to pull the guidewire through the blood outlet.

4. The percutaneous circulatory support system of claim 3, wherein the guidewire insertion aide comprises a proximal end portion and a distal end portion, and the distal end portion is disposed distally relatively to the blood inlet.

5. The percutaneous circulatory support system of claim 4, wherein the proximal end portion of the guidewire insertion aide is disposed outwardly from the blood outlet.

6. The percutaneous circulatory support system of claim 1, wherein the guidewire insertion aide lacks a through lumen.

7. A percutaneous circulatory support system, comprising:
a percutaneous circulatory support device, comprising:
a housing;
an impeller carried in the housing;
a guidewire insertion aide removably carried in the housing; and
a guidewire;
wherein the guidewire insertion aide and the guidewire are configured to be translatably coupled such that subsequent removal of the guidewire insertion aide from the housing causes the guidewire to be pulled through the housing.

8. The percutaneous circulatory support system of claim 7, wherein the percutaneous circulatory support device comprises a blood inlet and a blood outlet, and the guidewire insertion aide is configured to be removed from the housing through the blood outlet to pull the guidewire through the blood outlet.

9. The percutaneous circulatory support system of claim 8, wherein the guidewire insertion aide comprises a proximal end portion and a distal end portion, and the distal end portion is disposed distally relatively to the blood inlet.

10. The percutaneous circulatory support system of claim 8, wherein the proximal end portion of the guidewire insertion aide is disposed outwardly from the blood outlet.

11. The percutaneous circulatory support system of claim 7, wherein the guidewire insertion aide comprises a coupler for translatably coupling to the guidewire.

12. The percutaneous circulatory support system of claim 7, wherein the guidewire comprises a coupler for translatably coupling to the guidewire insertion aide.

13. The percutaneous circulatory support system of claim 7, wherein the guidewire insertion aide lacks a through lumen.

14. A method of manufacturing a percutaneous circulatory support system, the method comprising:
providing a percutaneous circulatory support device, the percutaneous circulatory support device comprising a housing and an impeller carried in the housing;
providing a guidewire insertion aide, the guidewire insertion aide being configured to be translatably secured to a guidewire; and
removably positioning the guidewire insertion aide in the housing of the percutaneous circulatory support device.

15. The method of claim 14, wherein the guidewire insertion aide comprises a coupler for translatably coupling to a guidewire.

16. The method of claim 14, wherein the guidewire insertion aide lacks a through lumen.

17. The method of claim 14, further comprising providing the guidewire detached from the guidewire insertion aide.

18. The method of claim 17, wherein the guidewire comprises a coupler for translatably coupling to the guidewire insertion aide.

19. The method of claim 14, wherein the percutaneous circulatory support device further comprises a cannula coupled to the housing, and further comprising removably positioning the guidewire insertion aide in the cannula.

20. The method of claim 19, wherein the percutaneous circulatory support device further comprises a flexible distal tip portion coupled to the cannula opposite the housing, and further comprising removably positioning the guidewire insertion aide in the flexible distal tip portion.
